(19) United States
(12) Patent Application Publication
(10) Pub. No.: US 2018/0296778 A1
(43) Pub. Date: Oct. 18, 2018

(54) VOLUME DISPLACEMENT DOSAGE VAPORIZER

(71) Applicant: Accugentix, LLC, Naples, FL (US)

(72) Inventor: Alainah M. Hacker, Naples, FL (US)

(21) Appl. No.: 15/951,651

(22) Filed: Apr. 12, 2018

Related U.S. Application Data
(60) Provisional application No. 62/484,879, filed on Apr. 12, 2017.

(51) Int. Cl. A61M 15/00; A61M 11/04

(52) U.S. Cl. CPC A61M 15/0066; A61M 15/002; A24F 47/008; A61M 11/042; A61M 15/0083

(57) ABSTRACT

A vaporizer device for the administration of a substance to a user as a vapor comprises a housing and a mechanism for delivering a vapor. The mechanism is located within the housing. The housing comprises one or more air intake openings, at least one air exit opening, at least one control button and a display screen. The mechanism for delivering a vapor comprises a translational screw in contact with a micro motor, a cavity adapted to engage a liquid-containing cartridge, a heating chamber adapted to receive a liquid from the liquid-containing cartridge, a heating element adapted to provide heat to the heating chamber, a first air channel in communication with the one or more air intake openings and the heating chamber, and a second air channel in communication with the heating chamber and the at least one air exit opening. A method of administering a vapor to an individual includes use of the vaporizer device where the vaporizer device is adapted to receive control instructions. A liquid-filled cartridge is provided which is adapted to be inserted into the cavity such that the cartridge can be placed in contact with the translational screw. The liquid-filled cartridge is inserted into the vaporizer; and a vapor provided by the vaporizer device is inhaled. The vaporizer device accurately determines the dosage that a patient is inhaling while also regulating their use of the device. This Volume Displacement Dosage Vaporizer is a reusable device with replaceable liquid cartridges. These cartridges can contain multiple dosages of a specific liquid, which is more convenient for the patient and reduces cost. Since the dosage is pre-measured while in liquid form, the air flow/suction force of the vapor does not affect the dosage. The device can also have locking mechanisms and wireless communication capabilities. The device will not allow patients take over the maximum doctor recommended dosage, and regulates the time interval of those dosages based on doctor recommendation. wireless communication can be used to communicate with other devices to ensure that the patient is not overlapping their dosages while using other devices. Wireless communication can also be used to sync the device to a smartphone, tablet, or computer app to track patient data, usage, and the patient can also set a timer/alarm.

### CROSS REFERENCE TO RELATED APPLICATIONS

This application claims benefit of pending U.S. Provisional Application No. 62/484,879 filed on Apr. 12, 2017.

### BACKGROUND OF THE INVENTION

This invention relates to vapor delivery systems. More particularly, this invention relates to vaporized drug delivery systems. Even more particularly, this invention relates to vaporized drug delivery systems which utilize volume displacement of a liquid which is then vaporized to provide a precise dosage of a vaporized substance.

Vaporizers are known in the art. Prior vaporizers have a general design that includes a battery, heating element, herb or liquid chamber, and a mouthpiece. Some vaporizers are desktop devices, while other devices are hand-held and portable. Some vaporizers have prepackaged single use doses, where the patient inserts the cartridge and uses for one session, and then has to dispose of it to insert a new cartridge. Other devices have prepackaged doses, and the device is able to check the air flow rate to determine the dosage to the patient. Still other devices use evaporation power and film thickness to determine a patient's specific dosage. Finally, some devices determine the dosage by the volumetric flow of vapor by using a volumetric flow controller.

These prior devices have inherent drawbacks. Devices using the flow rate of vapor do not measure the amount the direct dosage a patient or user intakes. Instead, they generally use indirect methods, like air flow rate and evaporation power to determine the dosage of a specific substance the user is inhaling. Using indirect methods is not accurate in its administration to the patient, and does not allow the patient to accurately track their usage. Also, some vaporizers use single-use cartridges with measured dosages, which are tedious to replace, and can be expensive for the consumer. Some vaporizers use cartridges containing herbal leaves, which makes it easier for users to extract and use outside of the device.

Therefore, there is a need for a vaporizer device that uses a mechanism that precisely and accurately determines the exact dosage a patient is inhaling. Such a device would enable patients and/or their doctors to track their usage and optimize treatment. There is also a need for a vaporizer device which can accurately administer a predetermined dosage without relying on single-use dosages.

### BRIEF SUMMARY OF INVENTION

It is, therefore, an aspect of the present invention to provide a reusable vaporizer. It is another aspect of the present invention to provide a reusable vaporizer that can be loaded with multiple doses at a time. In addition or in the alternative, the reusable vaporizer may be adapted to be used with different compounds that may be used concurrently. In one example, the vaporizer may administer a vaporized liquid substance. The liquid substance may be provided in a cartridge and different cartridges with different substances may be loaded and unloaded into the vaporizer. The patient may thereby administer a plurality of substances by interchanging cartridges in the vaporizer.

It is still another aspect of the present invention to provide an accurate pre-determined dosage. The vaporizer may be adapted to regulate the specific dosage of the predetermined substance administered to the patient. Additionally or in the alternative, the vaporizer may be adapted to regulate the time intervals between administration of the substance. In one such example, the vaporizer may be programmed by a medical professional to provide a maximum dosage of a substance over a predetermined time period. In this way, the device will not allow the patient to take more than needed and it will not allow the patient to use the device within a certain time frame.

In general, the present invention provides a medical device comprising a housing which surrounds and secures the mechanism of vapor delivery of the device. The housing includes one or more air intake openings and an air exit or mouthpiece opening for administration of the vaporized substance. The housing may include a power button to switch the device on or off, a display screen and control buttons entering information or altering the display provided on the display screen. Within the housing is a cavity adapted to receive a cartridge containing the substance to be delivered. The cartridge may be either reusable or disposable. With the cartridge installed, a plunger of the cartridge is adjacent to and is controlled by a translational screw, which can be turned by a micro motor. The micro motor is controlled by internal controls, described more fully below. The opposite end of the cavity for the cartridge is in communication with a heating chamber, which is heated by a heating element to a predetermined temperature, which is also controlled by the previously mentioned internal controls. The heating chamber is also in communication with the air intake openings and with the mouthpiece opening.

A valve may be present between in the air intake opening and the heating chamber. In addition or in the alternative, a valve may be present between the heating chamber and the air exit opening/mouthpiece. The air valves allow air flow in only one direction, and do not allow for vapor to leak. The air valves are opened from the force of the patient inhaling, and they close when that force of suction is gone. A power source such as a battery is provided to provide power to the heating element and to power the control elements which provide such functions as the amount of substance to be administered and the time interval between administrations. The power source may also provide power to optional components, such as wireless communication, a micro GPS chip, and password protection.

The device is adapted to receive control instructions which may be manually input or may be provided by a memory chip or card with the cartridge of the substance to be administered. The control instructions may include such information as the maximum dosage and minimum time interval between dosages prescribed or recommended by a doctor or other medical or professional personnel. The control instructions may also include information identifying the patient for whom the dosages are intended.

It is yet another aspect of the present invention to provide a method for administration of a substance to an individual. According to this method, a medical professional or other authorized technician may program a reusable vaporizer to deliver a specified amount of a predetermined substance per dosage and a minimum time interval between doses or other limiting parameters to assure that a maximum total dosage is not exceeded. This information may also be provided on a cartridge of the substance to be delivered by means of a memory chip which can be read by a memory chip reader present in the device. The substance to be administered is then loaded into the vaporizer device. In one example, the substance is provided in a cartridge or other similar container which is adapted to be inserted into the vaporizer. The vaporizer device and cartridge may be adapted to communicate such as via a memory card or chip in the cartridge and a memory card or chip reader in the vaporizer.

In further examples, when the cartridge is inserted into the device, the memory chip may relay information to the chip reader, including the type of (liquid) substance within the cartridge, the optimal temperature for that specific (liquid) substance to turn to vapor, and patient information, for example. Patient information may include a patient ID/tag, the recommended or prescribed dosages, and a recommended or prescribed time interval for those dosages.

The device may include a heating element. The heating element may be manually or automatically turned on once the device has received information about the identity of the substance, the optimal temperature of that substance, and patient information and dosage parameters. As the heating element preheats, the patient uses the interface of the device to choose their specific settings for that session of administration of the substance (also referred to as vaping). A specific dosage is chosen (less than or equal to the maximum doctor recommended or prescribed amount). The patient may choose a specific time interval to be notified about the next dosage (within doctor recommendation). The device will not allow the patient to choose any settings that are not within doctor recommended parameters. The patient finalizes their settings and can choose to save those settings for future use (“One Click Use”).

When the vaping session's settings are finalized, a micro motor turns a translational screw, which pushes forward a syringe plunger through the cartridge. Volume displacement within the cartridge is determined by the number of turns the translational screw makes. Each turn of the screw corresponds to a predetermined volume displacement within the cartridge. An encoder is used to monitor the actions of the motor and translational screw, and will stop the motor when the desired number of turns and/or partial turns are reached.

As the translational screw pushes the syringe plunger forward, liquid is dispensed from a small nozzle on the other end of the cartridge. This nozzle leads to the heating chamber, which is where the liquid stays until it becomes a vapor. The heating chamber does not directly touch the heating element, so there is a sensor within the chamber to notify the device when the optimal temperature is reached. The optimal temperature is where that specific liquid turns to vapor, so the heating chamber would then contain vapor, not liquid.

The device provides a signal to the patient that the device is ready for use, and the patient inhales. There are air channels for the entry point (below the heating chamber for example) and exit point (mouthpiece). There may be one or more air valves, such as one for the entry point and the other for the exit point. The air valves allow air flow in only one direction, and do not allow for vapor to leak. The air valves are opened from the force of the patient inhaling, and they close when that force of suction is gone.

Since the precise amount of medicine is measured while in liquid form, the patient only needs to inhale one or more times for the vapor to completely exit the device. When the patient is finished, they put the device to sleep or turn it off. If an alarm is set using the device, it may alert the user when the next dosage is available. The device will not work if the patient tries to use it before the specific time for their next dosage.

Optionally, the device may include various additional components, such as wireless communication, a micro GPS chip, and password protection. Wireless communication can be used sync data to the patient's computer, tablet, or smartphone app. This data could include: the exact dosage, time intervals of those dosages, and time of day of their vaping sessions, which can be used to track their use of the device. Wireless communication and app communication can be used to set up timers to notify the patient of their next dose. Wireless communication can also be used to communicate with other similar devices, preventing the patient from overusing their devices. The wireless communication between devices may also prevent overlap in the patient's dosages and time intervals. For security reasons, a micro GPS chip could be used to track a device if it is lost, and a password can be put on the device so that the patient would have to enter a short passcode before using the device.

### BRIEF DESCRIPTION OF THE SEVERAL VIEWS OF THE DRAWINGS

FIG. 1 is a perspective view of an example of the Volume Displacement Dosage Vaporizer of the present invention.

FIG. 2 is a partial cut-away view of an example of the vaporizer of the present invention in a static state, showing a full cartridge containing the substance to be delivered installed into the device.

FIG. 3 is a partial cut-away view of an example of the vaporizer of the present invention during use, showing a partially used cartridge containing the substance to be delivered installed into the device.

### DETAILED DESCRIPTION OF THE INVENTION

As stated above, the present invention is directed toward a reusable vaporizer that is adapted to deliver a predetermined amount of a substance to an individual. The invention may be understood more fully with reference to the following illustrative examples. These examples, however, should not be viewed as limiting the scope of the invention, but as a representative basis for understanding various aspects of the invention. The claims will serve to define the invention. It is to be understood therefore, that reusable vaporizer may vary in size and/or shaped depending upon the particular application it is designed for or even merely for aesthetic purposes. Likewise, differently configured arrangements of parts may fall within the scope of the present invention. For example, the examples shown in the drawings are not necessarily to scale and may vary in the configuration and/or placement of one or more components.

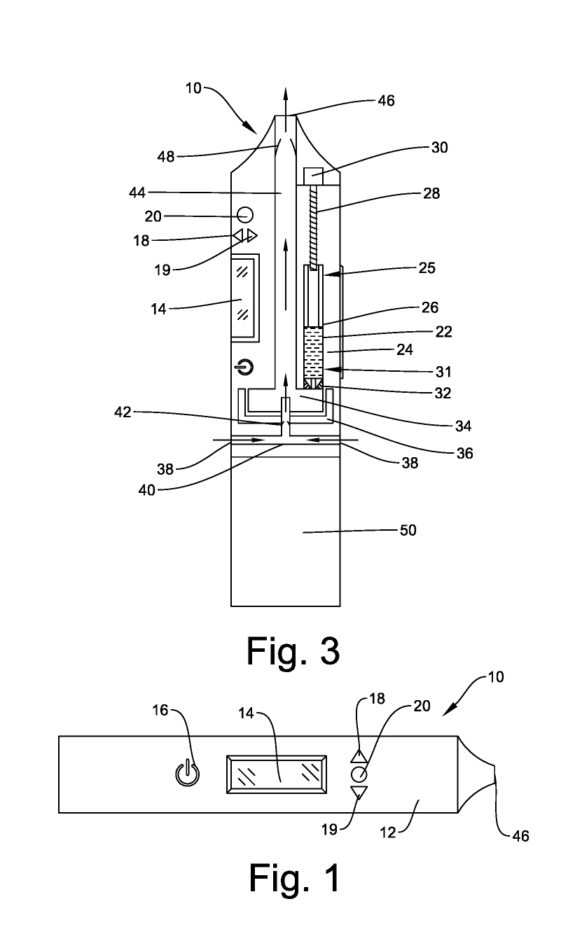

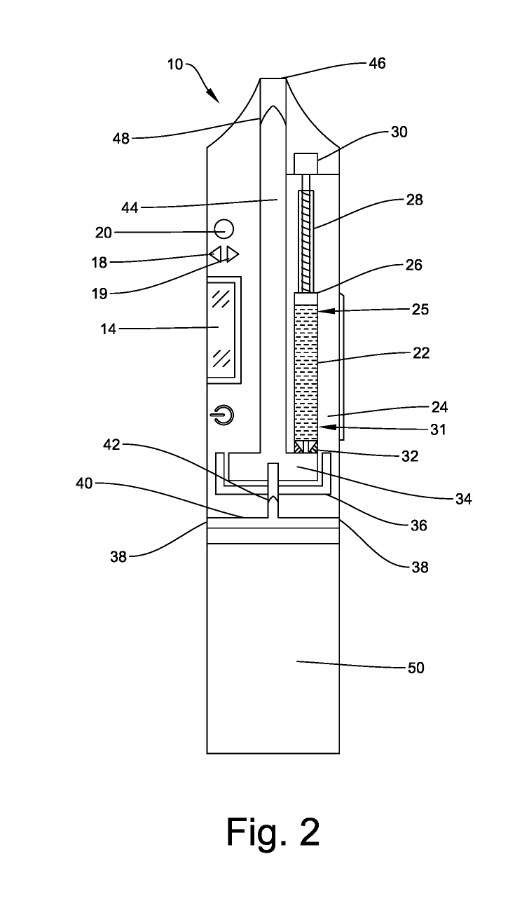

Referring to the drawings, wherein like reference numerals refer to like elements, one example of the present invention is shown in FIGS. 1-3. As shown in FIG. 1, the vaporizer device 10 comprises a housing 12 which surrounds and secures the mechanism of vapor delivery of the device. Housing 12 includes one or more air intake openings (38, shown in FIGS. 2 and 3) and an air exit aperture or mouthpiece opening 26. The housing may include a power button 16 to switch the device on or off, a display screen 14 and one or more control buttons (18, 19 and 20, respectively) for entering information or changing the display provided on display screen 14.

FIGS. 2 and 3 show a partial cut-away view of the vaporizer shown in FIG. 1. A cartridge 22 containing the liquid to be vaporized and administered to the individual is installed in a cavity 24 adapted to receive cartridge 22. Cartridge 22 may be reusable or disposable. Cartridge 22 has a first end 25 which includes a syringe type plunger 26. Plunger 26 can be extended into the interior of cartridge 22 by translational screw 28 which first end 25 contacts when engaged. Translational screw 28 in turn is controlled by micro-motor 30, which is activated and deactivated by internal control circuitry (not shown) to deliver a predetermined volume of the contents of cartridge 22 into heating chamber 34 as provide more fully below.

Cartridge 22 has a second end 31 opposite to first end 25 of cartridge 22. Second end 31 has an aperture 32 covered with a locking cap mechanism (not shown). Aperture 32 is in fluid communication with a heating chamber 34. Heating chamber 34 is in thermal contact with heating element 36. Heating chamber 34 is also in fluid communication with one or more air intake apertures 38 via first air channel 40. First air channel 40 may have a valve 42 located between aperture 38 and heating chamber 34. Valve 42 may be a one-way valve, allowing air to only pass from air intake aperture(s) 38 to heating chamber 34 and preventing vapor to escape from heating chamber 34.

Heating chamber 34 is also in fluid communication with air exit aperture or mouthpiece 46 via second air channel 44. Second air channel 44 may have a valve 48 located between heating chamber 34 and mouthpiece 46. Valve 48 may be a one-way valve, allowing air only to pass from heating chamber 34 to mouthpiece 46. As shown in FIG. 3, valves 42 and 48 open in response to inhalation by the individual, to allow air to pass from air intake apertures 38 to heating chamber 34 to mouthpiece 46.

The vaporizer further utilizes a battery 50 to power the internal circuitry (not shown), heating chamber 34, display 14, and any signals provided to the user, whether auditory, visual or tactical (such as by vibration). Battery 50 may be a rechargeable battery as known in the art.

Operation of vapor delivery device 10 may also be described with reference to FIGS. 1-3. The user may turn device 10 on by pressing power button 16 down for a predetermined period of time, such as 3-5 seconds. The user will have been previously provided with cartridge 22 adapted for use with device 10. Cartridge 22 may be reusable or disposable, and may be designed to contain multiple doses of liquid.

First end 25 of cartridge 22 contains a syringe type plunger 26, while second end 31 has a thin nozzle or aperture 32 covered with a locking cap mechanism (not shown) or other similar mechanism to prevent the contents of cartridge 22 to be extracted outside of the control of the vaporizer device 10. The cartridge may include a memory chip (not shown), which interacts with a memory chip reader (not shown) within the device 10 specifically for that patient. The memory chip on the cartridge can contain information about the substance within the cartridge, the optimal heat setting for that liquid to turn to vapor, the concentration of the liquid, and patient information. The patient information can include a specific ID/tag for that patient, a doctor recommended dose of medication, and a doctor recommended time frame for those dosages. The memory chip is programmed to notify the memory chip reader to unlock the locking cap mechanism.

The patient inserts cartridge 22 into device 10 (if first time use for that cartridge). The memory chip reader receives information from the memory chip on the cartridge. The memory chip informs the reader of the type of liquid within the cartridge, the optimal temperature for that liquid to turn to vapor, the concentration of the liquid, and patient information. Patient information received by the device includes a patient ID/tag, doctor recommended dosages, and doctor recommended time frame for those dosages. The device may be programmed to lock out the user if the specific patient ID/tag does not match the device (e.g. if the patient tries to use another patient's cartridge, another company's cartridge, a homemade cartridge).

Heating element 36 turns on to preheat the device (once the cartridge is securely in the device). Options on the interface/display screen 14 may appear, notifying the patient of what type and concentration of liquid in the cartridge. Depending on the contents, warnings about the substance may optionally appear. The patient can then choose a specific dosage using control buttons 18, 19, 20, but will not be able to go beyond the maximum recommended by their doctor. The patient can then choose a specific time frame for their next dosage as long as it is within their doctor's recommended time frame. The patient has the option to set a timer to notify them of next dosage. The patient finalizes their settings for that session and may have the option to save settings for future use (“One Click Use”).

Heating element 36 heats heating chamber 34 to a predetermined temperature. The temperature of heating chamber 34 is determined by a temperature sensor within the chamber (not shown). When heating chamber 34 reaches the predetermined temperature, the sensor and circuitry (such as a microprocessor) in device 10 maintain the temperature within heating chamber 34. The predetermined temperature may depend on the type of liquid within the chamber, which is correlated to the specific temperature at which the liquid turns to vapor.

When a predetermined temperature has been reached by heating chamber 34, micro motor 30 turns on, which turns a translational screw 28 or similar structure. Translational screw 28 engages cartridge 22 via plunger 26. It is envisioned that other configurations and structures of this mechanism may accomplish the same dosage. Micro motor 30 includes an encoder which monitors the rotations of translational screw 28 to determine the distance traveled by translational screw 28. The distance traveled is directly correlated to the volume displacement within cartridge 22. Variables of volume displacement include the diameter of the cartridge and the pitch of the screw. The encoder will stop micro motor 30 and translational screw 28 when the displacement of liquid within cartridge 22 equals the specific dosage.

As force of translational screw 28 pushes plunger 26 forward, the liquid within the cartridge is pushed through aperture 32 of second end 31 of cartridge 22. The liquid cannot escape/drip from the cartridge without the force of translational screw 28 and syringe plunger 26. The liquid aperture 32 continues into heating chamber 34, where the liquid turns to vapor. Heating chamber 34 is in thermal communication with heating element 36 but physical contact between heating chamber 34 and heating element 36 is not required. In some examples, it may be preferred that heating chamber 34 does not physically contact heating element 36.

When the volume displacement mechanism (i.e., plunger 26) is finished pushing the liquid into heating chamber 34 and when heating element 36 is maintained at the predetermined temperature, the patient may notified on by a message or signal on display 14 that the device is ready for use. Alternatively, the notification may be an auditory signal such as one or more tomes sounded, or a vibrational signal.

The patient inhales through mouthpiece 46 of device 10. There are air channels within the device to facilitate the flow of air as the patient inhales. One or more air intake apertures 38 are in fluid communication with heating chamber 34 via first air channel 40. First air channel 40 may have a valve 42 located between aperture(s) 38 and heating chamber 34. Valve 42 may be a one-way valve, allowing air to only pass from air intake aperture(s) 38 to heating chamber 34. When the patient inhales, valve 42 opens, permitting outside air to enter from apertures 38 and travel through first air channel 40 to enter heating chamber 34. The force of the individual's inhalation also opens valve 48, and air then flows through heating chamber 34 mixing with the vaporized liquid and passes to second air channel 44 and out at mouthpiece 46 of device 10, bringing the vapor with it.

In the example shown, the connected air channels 40, 44 are in fluid communication with one or more entering point(s)/apertures 38 (for outside air to enter the device) and have one exiting point (mouth piece 46). The entering point(s) of airflow may be positioned below the heating chamber and may lead to the main or second air channel 44 of device 10. Air goes through the heating chamber (where the vapor is), and continues to the exiting point/mouthpiece 46 through second channel 44.

In the example shown, device 10 includes two air valves 42, 48. A first valve 42 is positioned below heating chamber 34 and the other, 48, is positioned above heating chamber 34. The air valves 42, 48 are used to ensure that air/vapor cannot enter or exit the device without the force of suction (patient inhaling). The first air valve 42 positioned below the heating chamber prevents vapor from exiting the device, but ensures outside air enters the device when the patient inhales. The second air valve 48 above heating chamber 34 prevents outside air from entering device 10, but ensures the vapor exits device 10 when the patient inhales. Both air valves 42, 48 open when there is force of suction from the patient inhaling. After the patient stops inhaling, there is no force of suction, so air valves 42, 48 close. The air valves may be adjustable.

The present invention provides a precisely measured dosage before it is vaporized, so the patient only has to inhale one or more times to ensure the vapor has exited the device to obtain a well-defined dosage. After the patient has finished inhaling from the device, the power button may be pressed to put it to sleep/into a stand-by mode. If the patient does not put it to sleep, it may automatically go to sleep after a certain amount of time. If the patient set a timer for their next dosage, the device will wake up and an alarm will alert the patient to the availability of another dosage. When the patient is finished with the device, the device can be turned off fully by holding the power button for 3-5 seconds.

Optionally, display 14 may provide additional information such as remaining battery life and the amount of liquid left in the cartridge. The device (and other devices) may be programmed such that it cannot be used outside of the patient's doctor recommended time frame for the next dosage. This ensures the patient will not exceed their maximum doctor recommended dosage, which helps to prevent overdosing.

This device may also have wireless communication capabilities. Wireless communication can be used for this device to communicate with other corresponding devices. This ensures that the patient is not using more than their doctor recommended dosage and time frame. The wireless communication capabilities of this device can also be used to sync data with smartphone apps, tablet apps, or computer apps. Wireless communication can be used to manage the device from the patient's smartphone, tablet, or computer, allowing them to turn on/off the device and choose settings for the device. If the patient will use the same settings for that specific cartridge, the patient can save their settings in the app for “One Click Use” later on. Also, using wireless communication, the patient's smartphone, tablet, or computer app can notify them when they are allowed to use the device and can notify the patient for their next dosage (rather than using the device alarm).

Using a charger cord such as a USB cable and corresponding port for the battery, the patient can connect the vaporizer device to their computer manually. For security, the device also has the option to be password protected. In this case, the patient would have to type in a short password to use the device. It is also envisioned that the device can have a micro GPS chip. If the patient loses their device or if it is stolen, the device could be tracked using the existing GPS systems.

Based upon the foregoing disclosure, it should now be apparent that the volume displacement dosage vaporizer will carry out the objects set forth hereinabove. It is, therefore, to be understood that any variations evident fall within the scope of the claimed invention and thus, the selection of specific component elements can be determined without departing from the spirit of the invention herein disclosed and described.

I claim:

1. A vaporizer device for the administration of a substance to a user as a vapor, the vaporizer device comprising:
a housing;
a mechanism for delivering a vapor, wherein the mechanism is located within the housing;
wherein the housing comprises one or more air intake openings, at least one air exit opening, at least one control button and a display screen;
wherein the mechanism for delivering a vapor comprises a translational screw in contact with a micro motor, a cavity adapted to engage a liquid-containing cartridge, a heating chamber adapted to receive a liquid from the liquid-containing cartridge, a heating element adapted to provide heat to the heating chamber, a first air channel in communication with the one or more air intake openings and the heating chamber, and a second air channel in communication with the heating chamber and the at least one air exit opening.

2. The vaporizer device of claim 1, wherein at least one of the first air channel and the second air channel contains a one-way valve.

3. The vaporizer device of claim 2, wherein both the first air channel and the second air channel contain one-way valves.

4. The vaporizer device of claim 3, wherein the mechanism for delivering a vapor additionally comprises control circuitry.

5. The vaporizer of claim 4, wherein the control circuitry comprises a chip reader adapted to receive information from a chip provided with the liquid-containing cartridge.

6. The vaporizer device of claim 5, wherein the micro motor and heating element are controlled by control circuitry according to information provided by the chip reader.

7. The vaporizer device of claim 6, wherein the control circuitry establishes a maximum dosage and time intervals between dosages.

8. The vaporizer device of claim 7, wherein the maximum dosage and time intervals between dosages are outside of the control of the user of the device.

9. The vaporizer of claim 8, wherein the user may set a dosage less than the maximum dosage or greater than the minimum time interval between dosages.

10. A method of administration of a vapor to an individual, the method comprising:
providing a vaporizer device to the individual, wherein the vaporizer device comprises a housing and a mechanism for delivering a vapor, wherein the mechanism is located within the housing;
wherein the housing comprises one or more air intake openings, at least one air exit opening, at least one control button and a display screen;
wherein the mechanism for delivering a vapor comprises a translational screw in contact with a micro motor, a cavity adapted to engage a liquid-containing cartridge, a heating chamber adapted to receive a liquid from the liquid-containing cartridge, a heating element adapted to provide heat to the heating chamber, a first air channel in communication with the one or more air intake openings and the heating chamber, and a second air channel in communication with the heating chamber and the at least one air exit opening; and
wherein the vaporizer device is adapted to receive control instructions;
providing a liquid-filled cartridge adapted to be inserted into the cavity such that the cartridge can be placed in contact with the translational screw;
inserting the liquid-filled cartridge into the vaporizer; and
inhaling a vapor provided by the vaporizer device.

11. The method of claim 10, wherein vaporizer device comprises a chip reader and wherein the liquid-filled cartridge additionally comprises a memory chip adapted to carry control information and further wherein the control instructions are provided to the vaporizer device from the memory chip.

12. The method of claim 11, wherein the control instructions are provide by an authorized technician other than the user.

13. The method of claim 12, wherein the control instructions provide a maximum dosage and time intervals between dosages.

14. The method of claim 13, wherein the user may set a dosage less than the maximum dosage or greater than the minimum time interval between dosages.

15. The method of claim 14, wherein the vaporizer device is adapted to heat the heating chamber to a predetermined temperature when the vaporizer device is turned on in response to the control instructions.

16. The method of claim 15, wherein the micro motor is activated to dispense a predetermined amount of liquid from the liquid-filled cartridge to the heating chamber in response to the dosage and time interval set by the user, wherein the liquid is vaporized in the heating chamber.

17. The method of claim 14, wherein the micro motor is activated to dispense a predetermined amount of liquid from the liquid-filled cartridge in response to the dosage and time interval set by the user, wherein the liquid is vaporized in the heating chamber.

18. The method of claim 10, wherein at least one of the first air channel and the second air channel contains a one-way valve.

19. The method of claim 18, wherein the micro motor and heating element are controlled by the control instructions.

20. The method of claim 11, wherein the vaporizer device is additionally adapted to wirelessly communicate with other similar devices, a computer, a smartphone or a tablet.
